(19) United States
(12) Patent Application Publication
(10) Pub. No.: US 2006/0134313 A1
(43) Pub. Date: Jun. 22, 2006

(54) Methods for producing an anti-microbial plastic product

(76) Inventors: Josef-Peter Guggenbichler, Erlangen (DE); Christoph Cichos, Freiberg (DE)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 10/527,157

(22) PCT Filed: Sep. 10, 2003
(86) PCT No.: PCT/EP03/10049

§ 371 Date: Mar. 8, 2005

(30) Foreign Application Priority Data
Sep. 10, 2002 (DE) 102 41 962.0
Jul. 10, 2003 (DE) 103 31 324.9

(51) Int. Cl. A61L 33/00; B05D 3/00

(52) U.S. Cl. 427/2.1

(57) ABSTRACT

The invention relates to methods of producing metal-containing antimicrobial plastics or plastic products and also to plastic products obtainable by the method, especially plastic products for medical requirements.

The invention relates to methods of producing metal-containing antimicrobial plastic products and to products obtainable by the method, especially products for medical requirements.

Plastic articles are used in the medical field very frequently and for a very wide variety of purposes. A problem with the use of plastic products for medical purposes is the ease with which the plastics can be colonized by microbes. The microbes settle on the surface of the plastic and form a “biofilm”. Infections are a frequent consequence of using a plastic article colonized by microorganisms. It is known that the use of catheters and canulas made from plastics may easily result in infection due to inward migration of bacteria. Such infections are particularly serious and common in short-, medium- and long-term central venous catheters, among others, and also in the urological area, where urethral catheters and ureteral catheters are routinely used, and in the case of venticle drain systems. Thus in the Federal Republic of Germany alone each day approximately 12 to 15 patients die as a result of infections attributable to the use of microbially contaminated catheters.

Numerous attempts have been made to date to prevent the colonization of plastic articles and, consequently, infections. WO 87/03495 and WO 89/04682 describe the impregnation of medical devices and implants with antibiotics. A problem with antibiotic impregnation, however, is the development and selection of resistant microorganisms.

Another approach to reducing infections associated with the use of plastic products is the use of metals or metal alloys, e.g., for catheters (DE 40 41 721, DE 27 20 776 and DE 33 02 567). Of particular significance in this context is the antimicrobial property of silver. Silver and its salts, even in traces, exhibit a bacteriostatic and bactericidal action. U.S. Pat. No. 4,054,139 discloses a catheter in which for prophylaxis of infection a silver-containing, oligodynamic material has been applied to internal and external surfaces. In the approaches described, however, it has to date not been possible to achieve satisfactory results in any respect, particularly at the beginning of use, in respect of sterility with the impregnation of plastic products.

A method of producing antimicrobial plastic structures with improved long-term characteristics is described in WO 01/09229.

In a clinical trial of the catheters described in WO 01/09229 a reduction was observed in septic complications by 88% in relation to the infections caused by conventional catheters. This means that, in comparison to the use of control catheters, where 25 cases of sepsis occurred, the sepsis cases were reduced to three cases. Accordingly it is indeed the case that the action of a catheter produced by the method disclosed in WO 01/09229 is distinctly improved over the state of the art to that date; however, even with the use of the catheters disclosed in WO 01/09229, a colonization rate of 10% is observed, and in that case as well, moreover, particularly in the first few days following implantation of the catheter, there were infections at the entry site of the catheter.

Accordingly it has been impossible to date to prevent microbial contamination of plastic products used medically, particularly of catheters, to a satisfactory extent.

The object of the present invention is therefore to provide a method of producing plastic products which exhibit satisfactory antimicrobial activity.

This object is achieved by means of a method of producing an antimicrobial plastic product, comprising
A) forming an intermediate product,
B) treating at least one constituent of the intermediate product with an antimicrobial metal colloid, and
C) adding a readily or sparingly soluble salt of an antimicrobial metal.

Surprisingly the combination of an antimicrobial metal colloid and a readily or, preferably, sparingly soluble salt of an antimicrobial metal produces a satisfactory antimicrobial activity. In addition to a sufficient long-term action, a distinctly improved immediate action against microorganisms as well is achieved with the plastic product of the invention. In particular the antimicrobial activity at the beginning is substantially improved as compared with a prior art plastic product as described in WO 01/09229, for example. Thus, in a direct comparison of the plastic products produced according to WO 01/09229 with the plastic products of the invention, it is possible to show a significantly higher antimicrobial activity on the part of the plastic products of the invention (cf. table 1).

The plastic products according to the present invention, moreover, do not possess increased cytotoxicity as compared with prior art products; a further advantage is that when the plastic products of the invention are used no thrombogenicity is observed.

Antimicrobial plastic products for the purposes of the invention are products which exhibit activity against microorganisms, particularly against bacteria and/or fungi. The action in question may comprise both a bacteriostatic action and a bactericidal action.

By means of the method of the invention it is possible in principle to produce any desired antimicrobial plastic product, preference being given to producing products which find use in the medical sector. These may be, for example, catheters, hoses, tubes, especially endotracheal tubes, articles used in urology, bone cement, preferably methyl acrylate bone cement, Goretex fabric, toothbrushes, silicone plastics, polymeric films, textiles, for occupational apparel for example, diapers and/or parts thereof. In one particularly preferred embodiment of the method of the invention catheters are produced.

As starting materials for producing the antimicrobial plastic products of the invention it is possible to employ any desired polymeric compounds which commonly find use in the medical sector. Preferred polymers are, for example, polyurethanes, polyethylene, polypropylene, crosslinked polysiloxanes, (meth)acrylate-based polymers, cellulose and its derivatives, polycarbonates, ABS, tetrafluoroethylene polymers, polyethylene terephthalates, and the corresponding copolymers. Particular preference is given to the use of polyurethane, polyethylene and polypropylene and also of polyethylene/polypropylene copolymers, with polyurethane being the most preferred.

In addition to one or more polymeric materials the intermediate product may comprise further additives. Additives can be, for example, organic or inorganic substances. The intermediate product may comprise any organic and inorganic substances which are inert and medically unobjectionable, such as, for example, barium sulfate, calcium sulfate, strontium sulfate, titanium dioxide, aluminum oxide, silicon dioxide, zeolites, calcium fluoride (CaF₂), mica, talc, pyrogenic silica, calcium hydroxylapatite, kaolin, zirconium and/or microcellulose. Inorganic substances used with preference are barium sulfate, which for certain forms of application can be used simultaneously as an x-ray contrast medium, and zirconium.

In the method of the invention one or more constituents of the intermediate products are treated with a metal colloid. In this context it is possible to treat one or more polymeric materials and/or one or more organic and/or inorganic particles with the metal colloid. The support materials for the metal colloid may be present in the intermediate product in an amount of from about 5 to 50% by weight. If barium sulfate is used as support material it is customarily present in an amount of from about 5 to 30% by weight, with particular preference in an amount of about 20% by weight. Where silicon dioxide is used as support material it is present in an amount of from about 30 to 50% by weight, preferably about 40% by weight.

The metal colloid which can be used to treat one or more constituents of the intermediate product is suitably prepared by reduction of metal salt solutions. Where silver is used, it is admixed with a reducing agent, the silver being in the form, for example, of ammoniacal silver nitrate solution. To stabilize the resultant metal colloid it is additionally possible if desired to use protective substances such as gelatin, silica, starch, dextrin, gum arabic, polyvinyl alcohol or complexing agents such as ethylenediaminetetraacetic acid. It is preferred to operate without protective substances. Examples of suitable reducing agents are aldehydes (e.g., acetaldehyde), aldoses (e.g., glucose), quinones (e.g., hydroquinone), complex inorganic hydrides (sodium or potassium boronate), reducing nitrogen compounds (e.g., hydrazine, polyethylenimine), ascorbic acid, tartaric acid and citric acid.

By varying the reducing agents and by varying or omitting the stabilizers it is additionally possible to control the coloring of the coated support material.

All metals having an antimicrobial action are suitable for the method of the invention, such as, for example, silver, copper, gold, zinc, zirconium, bismuth or cerium and also mixtures thereof. Particular preference is given to silver, which has a high antimicrobial activity. Copper as well is used with preference, and its use advantageously achieves activity with respect to fungi as well.

The amount of the metal colloid is advantageously from about 0.1 to 10%, preferably from about 0.5 to 5% by weight.

The application of the metal colloid to one or more constituents of the intermediate product can take place either in one step or can be followed by drying and repeated a number of times. Both techniques can be used to achieve a very high metal concentration. By varying the reducing agents and by varying or omitting the stabilizers it is possible to control the particle size of the metal. If silver is used as the metal colloid, the preferred particle size is in the range from 10 to 50 nm. Silver of this particle size is referred to as nanosilver. In one preferred embodiment the addition of the reducing agent and the deposition of the nanosilver is followed by the precipitation, by addition of phosphoric acid, of the silver that has remained in the solution, the precipitated silver being in the form of silver phosphate, which is referred to below as “silver phosphate in the nascent state” and is distinguished by particularly rapid onset of the antimicrobial action.

The amount of the metal colloid is chosen so that a sufficient portion of the surface of the plastic product is composed of metal particles in order to achieve an antimicrobial activity.

In accordance with the invention a readily soluble or sparingly soluble salt of an antimicrobial metal is additionally added to the intermediate product. This salt preferably comprises a silver salt, zinc salt, copper salt, cerium salt, platinum salt, zirconium salt, bismuth salt and/or gold salt and also mixtures thereof. Particular preference is given to using a silver salt, especially silver sulfate and/or silver phosphate in the nascent state. In principle suitability is possessed by any readily or sparingly soluble salts of antimicrobially active metals that are stable to exposure to light and are physiologically unobjectionable. The amount of the metal salt used can be from 0.1 to 5% by weight, based on the total weight of the intermediate product, preferably from 0.5 to 1% by weight.

After the constituents of the intermediate product at least partly treated with a metal colloid have been mixed with the sparingly soluble metal salt, the mixture obtained is processed further to give a plastic product. This can be done, for example, by extruding, injection molding, mixing, kneading or (hot) pressing. Preferred shaping processes are extrusion and injection molding.

The present invention further provides plastic products obtainable by the method of the invention. The plastic products in question are preferably products which find use in the medical sector. In one particularly preferred embodiment the method of the invention is used to produce catheters.

Examples of the preferred medical products are venous catheters for short-term implantation, where not only the outside of the catheter but also each lumen inside, the Luer lock and the manifold are made of the material obtained in accordance with the invention. Experiments have shown that an inoculum size of 10⁹ microbes, used to contaminate the surface, is eliminated completely within less than 9 hours. Additionally there are peripheral venous canulas, Sheldon catheters for implantation over 6 weeks for hemodialysis, Hickman-type catheters for long-term implantation, with a cuff made from material produced in accordance with the invention (antimicrobial activity of at least one year ascertained), port catheters, where at least the port chamber is made from material produced in accordance with the invention, and advantageously all other constituents thereof, ventricular drain catheters (minimum period of activity 3 years), bladder catheters, cystostomy, nephrostomy catheters, urether stents (e.g., of polyurethane or silicone base material; advantageously the entire urine collection system and the connectors are composed of said material), thorax drains and the attached suction system, endotracheal tubes, Tenckhof catheters with cuff, bone cements (based on methyl acrylate, for example), toothbrushes (bristles and handle), surgical suture material, filament material for producing antimicrobial textiles, coating materials for antimicrobial coating, e.g., of hoses for ventilation, antimicrobial wound coverings and dressings in the event of burn injuries.

In the text below a description is given of preferred embodiments of the method of the invention.

In one preferred embodiment polyurethane pellets with a size of approximately 1 mm³ are used as polymeric material. A further constituent of the intermediate product is barium sulfate, which functions as support material. Deposited on the barium sulfate are about 3 to 10% by weight, or even more if desired, of nanosilver. The intermediate product additionally includes about 0.5 to 1% by weight of silver sulfate or silver phosphate, particularly in the nascent state. The constituents of the intermediate product are mixed; further processing can take place by extrusion.

In another preferred embodiment the metal salt used comprises a combination of silver and copper in a silver/copper ratio of about 2:1. This combination advantageously also possesses a satisfactory microbial activity against fungi.

According to another preferred embodiment a combination of a metal colloid, with particular preference nanosilver, and zirconium silicate is used. Particularly suitable are silver to zirconium silicate weight ratios of 1:1-10.

The invention is further illustrated by the figures and examples below.

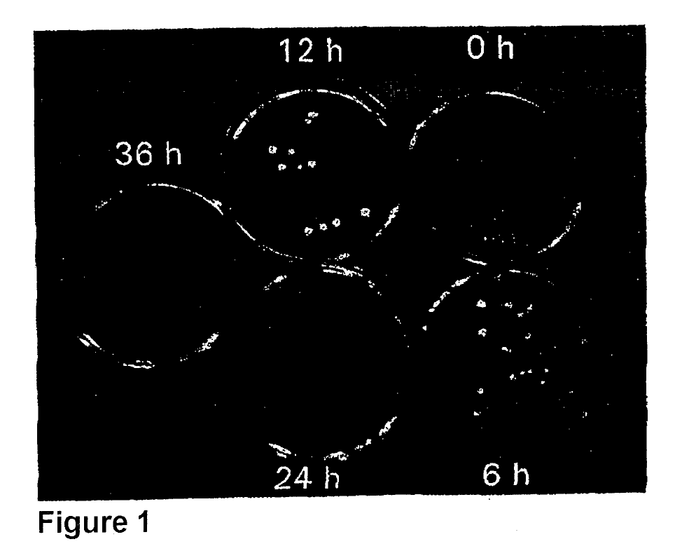

FIGS. 1 to 3 show results of experiments relating to antimicrobial activity. The microorganism used was in each case Staphylococcus epidermidis ATCC 14 990, with a starting microbe count of 5×10⁷ CFU/ml.

In the experiment shown in FIG. 1, 0.8% of nanosilver and 0.5% of silver sulfate were used.

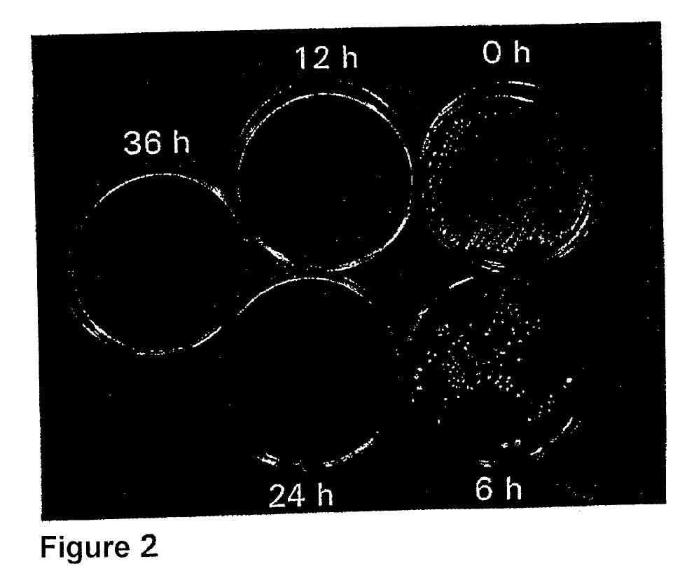

In the experiment shown in FIG. 2, 0.8% of nanosilver and 1.0% of silver sulfate were used.

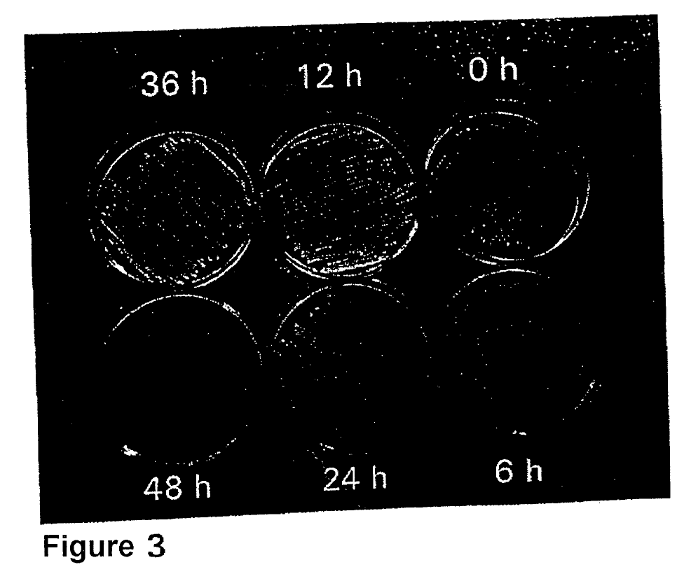

FIG. 3 shows an experiment in which 0.8% of nanosilver and no additional silver sulfate was used.

### EXAMPLES

### Comparative Example 1

### Commercially Customary Plastic According to WO 01/09299

A: Preparation of a Silver Colloid

1.0 g (5.88 mmol) of AgNO₃ p.a. are dissolved in 100 ml of distilled water and the solution is admixed with 1.0 ml (14.71 mmol) of 25% strength aqueous NH₃. To prepare the silver colloid, a solution of 258.7 mg (5.88 mmol, 330 μl) of acetaldehyde in 50 ml of distilled water is added slowly dropwise to the first solution over a period of 30 minutes at 40° C.

B: Coating of Polyurethane Pellets

10 minutes after the end of the dropwise addition as described in example 1 about 50 g of polyurethane pellets of Tecothane TT-1085A are added and for coating with colloidal silver are stirred vigorously at 40° C. for 2 hours to start with and then at room temperature for 3 hours. The silver colloid is separated off by rapid filtration through a fluted filter of appropriate pore size, and the pellets are washed again with the filtrate and, while still moist, are transferred to an evaporation boat. After the removal of excess silver colloid solution, not adhering to the polymer, drying takes place at 70° C. for 10 hours.

### Example 2

### Plastic with Improved Antimicrobial Activity

A: Adsorption of Colloidal Silver on Barium Sulfate

The following are dissolved in succession in 360 ml of distilled water at 50° C.: 0.6 g of gelatin and 6.0 g of AgNO₃. 7.8 ml of 25% strength aqueous ammonia solution are added to the first solution. With vigorous stirring at 50° C. a solution of 3.18 g of anhydrous glucose in solution in 120 ml of distilled water is metered in slowly. When approximately half the amount of glucose has been added dropwise, 100 g of barium sulfate is introduced with vigorous stirring into the silver colloid already formed, and the addition of glucose is continued. When the addition of glucose is at an end the suspension is agitated with a turbine stirrer for a further 2 hours, initially at 50° C., and thereafter at 70° C. for 3 hours.

Subsequently the solid is separated from the liquid by filtration or centrifugation. The solid is washed repeatedly with ultra-pure water until free of electrolyte, and is filtered, dried at 70° C. to 80° C. and finely comminuted.

B: Admixing of Silver Sulfate

The dried and comminuted barium sulfate is admixed with 2.5% by weight or 5% by weight of finely ground silver sulfate and the two components are mixed thoroughly.

C: Mixing of the Individual Constituents

20% by weight of the coated barium sulfate/silver sulfate mixture are mixed thoroughly with 77.6% by weight of polyurethane pellets and 2.4% by weight of a further, inorganic, uncoated material, e.g., titanium dioxide, and the mixture is subjected to a further operation, e.g., an extrusion.

If 2.5% by weight of silver sulfate are added in step B, the plastic set out under A in table 1 is obtained; if 5% by weight of silver sulfate are added in step B, the plastic set out under B in table 1 is obtained.

### Example 3

### Plastic with Improved Antimicrobial Activity

A: Adsorption of Colloidal Silver on Barium Sulfate

18 g of AgNO₃ are dissolved in 1080 ml of distilled water at 50° C. and 200 g of barium sulfate are added. The suspension is stirred vigorously for about 20 minutes and thereafter is admixed with 23.4 ml of 25% strength aqueous ammonia solution.

With continual stirring, and with the temperature remaining the same, 9.6 g of anhydrous glucose in solution in 360 ml are slowly added dropwise. After the end of the addition of glucose, the procedure continues in the same way as in example 2A up to the point of the grinding of the dried barium sulfate.

B: Admixing of Silver Sulfate

The admixing of silver sulfate takes place in the same way as in example 2B.

C: Mixing of the Individual Constituents

In the same way as in example 2 the barium sulfate mixture of silver sulfate is mixed with the other constituents and subjected to further processing.

### Example 4

### Determination of the Antimicrobial Activity

The antimicrobial activity of the plastics of the invention was determined by incubating samples of the plastics in question with a trypcase-soy broth nutrient solution containing different microbes at 37° C.

Microorganisms Used:

Staphylococcus epidermidis (S. epidermidis) ATCC 14 990, S. epidermidis, fresh clinical isolate from a patient with catheter-associated sepsis,

Staphylococcus aureus (S. aureus) ATCC 25923,

Escherichia coli (E. coli), fresh clinical isolate from a patient with catheter associated sepsis,

Pseudomonas aeruginosa (P. aeruginosa), fresh clinical isolate from a patient with catheter-associated sepsis.

The microbe count was adjusted in a photometer either to 5×10⁷ colony forming units (CFU)/ml (corresponding in the case of Staphylococci to an OD of 0.30 at 457 nm, in the case of P. aeruginosa and E. coli to an OD of 0.65) or 10⁹ CFU/ml (OD 0.65 for staphylococci at 475 nm, 1.2 for P. aeruginosa and E. coli). Determination of the CFU/ml was carried out in parallel by serial dilution on agar plates, and the microbe counts determined by photometric measurement were confirmed.

Plastic Materials:

Polyurethane (Tecoflex) was used, a material from which virtually all implantable central venous catheters are manufactured. This material was coextruded with nanosilver (particle size 3 to 5 nm) in an amount of 0.8% or 1.3% by weight and with different concentrations of silver sulfate (0.25%, 0.5%, 0.75% and 1.0%). Extrudates with an external diameter of 1.6 mm were manufactured. From these extrudates, pellets each 1 mm in length were chopped, with 10 pellets giving a surface area of approximately 1 cm² and 50 pellets a surface area of 5 cm².

Test Method:

The sections of plastic (with a surface area of either 1 cm² or 5 cm⁵) were introduced into a suspension containing either 5×10⁷ CFU/ml or 10⁹ CFU/ml of the above-described microbes in physiological saline solution. The test specimens were shaken at a speed of 120 rotations/minute. At the beginning of the investigation (starting microbe count) and after 6, 12, (18), 24, 36 and (48) hours in each case 1 loop (2 μl) was removed and plated out on agar (Müller Hinton agar). The plates were incubated at 37° C. for 24 hours. Subsequently the microbe count on the agar plate was determined by counting the colonies.

All of the experiments were repeated three times, with the data below representing in each case the mean values of the three corresponding experiments.

Results:

Table 1 below lists the colony counts found for the test experiment, as obtained with S. epidermidis ATCC 14 990.

TABLE 1

| | | | | | | |
|---|---|---|---|---|---|---|
| Time in hours | 0 | 6 | 12 | 24 | 36 | 48 |

A 0.8% nanosilver, 0.5% silver sulfate

| | | | | | | |
|---|---|---|---|---|---|---|
| 5 × 10⁷ CFU/ml | | | | | | |
| 1 cm²* | 5 × 10⁷ | 2 × 10³ | 10³ | 0 | 0 | — |
| 5 cm² | 5 × 10⁷ | 10³ | 0 | 0 | 0 | — |
| 10⁹ CFU | | | | | | |
| 1 cm² | 10⁹ | 10⁷ | 0 | 0 | 0 | — |
| 5 cm² | 10⁹ | 10⁵ | 0 | 0 | 0 | — |

B 0.8% nanosilver, 1.0% silver sulfate

| | | | | | | |
|---|---|---|---|---|---|---|
| 5 × 10⁷ CFU/ml | | | | | | |
| 1 cm² | 5 × 10⁷ | 10⁴ | 0 | 0 | 0 | — |
| 5 cm² | 5 × 10⁷ | 10³ | 0 | 0 | 0 | — |
| 10⁹ CFU | | | | | | |
| 1 cm² | 10⁹ | 10⁶ | 10² | 0 | 0 | — |
| 5 cm²** | 10⁹ | 10⁴ | 0 | 0 | 0 | — |

C 0.8 nanosilver
(commercially customary plastic according to WO 01/09229; Medex)

| | | | | | | |
|---|---|---|---|---|---|---|
| 5 × 10⁷ CFU/ml | | | | | | |
| 1 cm² | 5 × 10⁷ | 10⁷ | 10⁶ | 10⁴ | 10³ | 0 |
| 5 cm² | 5 × 10⁷ | 10⁶ | 10⁵ | 10³ | 10² | 0 |
| 10⁹ CFU | | | | | | |
| 1 cm² | 10⁹ | 10⁹ | 10⁹ | 10⁹ | 10⁹ | 10⁸⁺ |
| 5 cm²*** | 10⁹ | 10⁹ | 10⁹ | 10⁹ | 10⁹ | 10⁶⁺ |

*weak growth of the colonies after 48 hours' incubation
shown in FIG. 1
shown in FIG. 2
shown in FIG. 3

A corresponding growth behavior is also shown by the wild strain of S. epidermidis, S. aureus ATCC 25923, and E. coli and P. aeruginosa. The test experiments showed that the addition of silver sulfate significantly increases the immediate antimicrobial activity (comparison of A or B with C). The increase in the activity as a result of adding silver sulfate is dose-dependent, but an activity can be observed even with an addition of 0.5% of silver sulfate. The plastic of the invention exhibits a significantly improved antimicrobial activity in comparison with a plastic containing only nanosilver (experiment C). In the case of the prior art plastic tested (from WO 01/09229) sterility can be observed only after 48 hours with a starting microbe count of 5×10⁷ of CFU/ml. With a starting microbe count of 10⁹ CFU/ml there is still weak growth of the colonies even after 48 hours.

### Example 5

Investigation of Support Material Containing Zirconium Silicate

The barium sulfate support material is admixed in a first series of experiments with 20% by weight of zirconium silicate, in a second series of experiments with 20% by weight of nanosilver and 20% by weight of zirconium silicate. The resulting mixtures are admixed with different quantities of microbes and then the microbial growth is recorded over 48 hours.

| | |
|---|---|
| Zirconium silicate without | Zirconium silicate with |
| silver - microbe | nanosilver - microbe |
| count/ml | count/ml |

| | | | | | | | | |
|---|---|---|---|---|---|---|---|---|
| Time | 10⁹ | 10⁸ | 10⁷ | 10⁶ | 10⁹ | 10⁸ | 10⁷ | 10⁶ |

| | | | | | | | | |
|---|---|---|---|---|---|---|---|---|
| 2 h | + | + | + | − | + | +/− | − | − |
| 3 h | + | + | + | − | + | +/− | − | − |
| 6 h | + | + | +/− | − | + | − | − | − |
| 12 h | + | +/− | − | − | + | − | − | − |
| 18 h | + | +/− | − | − | + | − | − | − |
| 24 h | + | − | − | − | + | − | − | − |
| 30 h | + | − | − | − | + | − | − | − |
| 36 h | + | − | − | − | + | − | − | − |
| 42 h | + | − | − | − | +/− | − | − | − |
| 48 h | + | − | − | − | − | − | − | − |

+ = growth
− = sterile
+/− = no growth but also still not sterile

### Example 6

Comparative Investigation of the Antimicrobial Activity of Zirconium Silicate on Barium Sulfate as Support Alone or with Nanosilver

| Time (h) | 1 | 2 | 3 | 4 | 6 | 9 | 12 |
|---|---|---|---|---|---|---|---|
| Control (microbe count/ml) | 10⁹ | 10⁹ | 10⁹ | 10⁹ | 10⁹ | 10⁹ | 10⁹ |
| 1% zirconium silicate on barium | 10⁹ | 10⁸ | 10⁸ | 10⁷ | 10⁷ | 10⁶ | 10⁵ |
| sulphate | | | | | | | |
| 0.1% zirconium silicate on barium | 10⁹ | 10⁹ | 10⁹ | 10⁹ | 10⁹ | 10⁹ | 10⁹ |
| sulphate | | | | | | | |
| 1% zirconium silicate + 5% | 10⁸ | 10⁶ | — | — | — | — | — |
| nanosilver on barium sulfate | | | | | | | |
| 0.1% zirconium silicate + 5% | 10⁹ | 10⁷ | 10⁵ | — | — | — | — |
| nanosilver on barium sulfate | | | | | | | |
| 1% zirconium silicate + 3.5% | 10⁹ | 10⁸ | 10⁶ | — | — | — | — |
| nanosilver on BaSO₄ | | | | | | | |
| 0.1% zirconium silicate + 3.5% | 10⁹ | 10⁹ | 10⁷ | 10⁶ | 10⁶ | 10⁵ | — |
| nanosilver on barium sulfate | | | | | | | |

### Example 7

Investigation of the Antimicrobial Activity when Using Nanosilver and Silver Phosphate in the Nascent State on Barium Sulfate Support (3.6% Ag; 5% Silver Phosphate

Adsorption of colloidal silver on barium sulfate and generation of ultrafinely divided silver phosphate in the nascent state

14.45 g of silver nitrate are dissolved in 360 ml of distilled water at 50° C. and then with vigorous stirring 100 g of barium sulfate are introduced. The suspension is stirred for about 20 minutes. Thereafter 19.3 ml of a 25% strength aqueous ammonia solution are added.

With continual stirring and with the temperature remaining the same, a solution of 5.25 g of glucose monohydrate in 182 ml of distilled water is metered slowly into the suspension. After the end of the addition of glucose stirring is continued for 2 to 4 hours more at 50° C. and finally the nonreduced silver still present is precipitated with about 50 ml of 0.1 molar phosphoric acid and the suspension is brought to a pH of approximately 6.

Stirring is continued until the suspension has cooled to room temperature. Subsequently the solid is separated off by sedimentation, filtration or centrifugation.

The resulting solid is washed repeatedly with ultrapure water until free of electrolyte and finally is dried at 70 to 80° C. in a drying cabinet and, if desired, is comminuted after drying.

The product produced in this way is whitish gray in color; its composition is 3.6% nanosilver, 5% silver phosphate on BaSO₄. The microbe count at a concentration of 1% or 0.1% was determined in accordance with example 4:

| Time (h) | 1 | 2 | 3 |
|---|---|---|---|
| 1% | 10⁷ | 10⁵ | |
| 0.1 | 10⁸ | 10⁷ | 10⁶ |

### Example 8

A: Adsorption of Colloidal Silver on Barium Sulfate

9 g of silver nitrate are dissolved in 360 ml of distilled water heated to 50° C., and with vigorous stirring 100 g of barium sulfate are introduced. After 20 minutes of stirring 12 ml of a 25% strength ammonia solution are added.

Subsequently, with the temperature remaining the same, a solution of 5.25 g of glucose monohydrate in 182 ml of distilled water is metered in slowly. After the end of the addition of glucose the suspension is stirred at 50° C. for a further 2 to 4 hours and then at 70° C. for 1 to 3 hours.

After complete reaction the solid is separated from the aqueous phase and washed repeatedly with ultrapure water or distilled water until free of electrolyte. The washed solid is dried at 70 to 80° C. in a drying cabinet and thereafter comminuted to the primary particle size.

B: Admixing of Silver Phosphate

The desired amount (1 to 5% by weight) of ultrapure silver phosphate is added to the solid obtained according to A and the two components are mixed thoroughly. Investigation as described in example 4 gave results of a similarly good quality to those shown in example 7.

1. A method of producing an antimicrobial plastic product, comprising
A) forming an intermediate product,
B) treating at least one constituent of the intermediate product with an antimicrobial colloidal metal, and
C) adding a readily or sparingly soluble salt of an antimicrobial metal.

2. The method of claim 18, characterized in that the sparingly soluble metal salt is selected from the group consisting of silver salts, zinc salts, copper salts, cerium salts, zirconium salts, bismuth salts, platinum salts and/or gold salts.

3. The method of claim 2, characterized in that the metal salt comprises silver sulfate and/or silver phosphate.

4. The method of claim 3, characterized in that the metal salt is present in an amount of from 0.1 to 1.0% by weight, based on the total weight of the intermediate product.

5. The method of claim 2, characterized in that the metal salt is present in a silver/copper ratio of approximately 2:1.

6. The method of claim 1, characterized in that the intermediate product comprises one or more polymeric materials.

7. The method of claim 6, characterized in that the intermediate product comprises polyurethane.

8. The method of claim 6, characterized in that the intermediate product comprises further additives.

9. The method of claim 8, characterized in that the additives comprise organic and/or inorganic particles.

10. The method of claim 9, characterized in that the organic and/or inorganic particles are selected from the group consisting of barium sulfate, calcium sulfate, strontium sulfate, titanium dioxide, aluminum oxide, silicon dioxide, zeolites, calcium fluoride (CaF₂), mica, talc, pyrogenic silica, calcium hydroxylapatite, kaolin and/or microcellulose.

11. The method of claim 8, characterized in that the additives comprise inorganic particles that comprise barium sulfate and/or pyrogenic silica.

12. The method of claim 9, characterized in that the polymeric materials and inorganic particles are treated with a colloidal metal.

13. The method of claim 1, characterized in that the constituent of the intermediate product that is inorganic particles are treated with a colloidal metal comprises inorganic particles.

14. The method of claim 1, characterized in that the colloidal metal comprises colloidal silver.

15. The method of claim 1, characterized in that the mixture of treated intermediate product and sparingly soluble metal salt is shaped by extruding, injection molding, mixing, kneading or (hot) pressing.

16. A plastic product made by the process of claim 1.

17. The plastic product of claim 16 in the form of a catheter.

18. The method of claim 1, characterized in that the metal salt is a sparingly soluble salt of an antimicrobial metal.

19. The method of claim 1, characterized in that the colloidal metal is nanosilver, and metal salt is zirconium silicate.

20. The method of claim 19, characterized in that the silver to zirconium silicate weight ratio is 1:1-10.
